(19) United States
(12) Patent Application Publication
(10) Pub. No.: US 2010/0106077 A1
(43) Pub. Date: Apr. 29, 2010

(54) Methods, Compositions and Apparatus for Treating a Scalp

(75) Inventors: Michael I. Rabin, Gates Mills, OH (US); David A. Smith, Gates Mills, OH (US)

Correspondence Address: CROCKETT & CROCKETT, P.C., 26020 ACERO, SUITE 200, MISSION VIEJO, CA, 92691, US

(73) Assignee: Transdermal Cap, Inc.

(21) Appl. No.: 12/604,892

(22) Filed: Oct. 23, 2009

Related U.S. Application Data
(63) Continuation-in-part of application No. PCT/US08/61350, filed on Apr. 23, 2008.
(60) Provisional application No. 60/913,532, filed on Apr. 23, 2007.

(51) Int. Cl. A61M 35/00; A61N 5/06

(52) U.S. Cl. 604/20; 607/91

(57) ABSTRACT

A phototherapy light cap is a flexible, generally hemispherical cap having a light source to supply suitable dosage requirements of current and future light therapies. The phototherapy light cap may also include a rechargeable battery source, a light source or an array of light sources, a light diffuser and an interface to a recharging source that may be a docking station. A phototherapy light cap may alternatively be an insert for any commercial head dressing, preferably adapted for convenient recharging. Combining the use of a phototherapy light cap with gelatinized therapeutic agents provides a suitable treatment technique for a scalp utilizing phototherapy, heat and any suitable combination of active ingredients such as minoxidil.

### RELATED APPLICATIONS

This application is a continuation-in-part of PCT application PCT/US08/61350 filed Apr. 23, 2008 which claims priority from U.S. Provisional patent application 60/913,532 filed Apr. 23, 2007.

### FIELD OF THE INVENTIONS

The inventions described below relate to the field of hair growth and regeneration in a human scalp.

### BACKGROUND OF THE INVENTIONS

There is a substantial body of anecdotal evidence supporting phototherapy for promoting human hair growth and regrowth. Additional evidence exists that low-level light therapy (LLLT) may be most beneficial if provided within one or more narrow spectral windows.

At least three US manufacturers sell products that deliver red light to the scalp: Sunetics, HairMax and Laser Hair Therapy. Prior art methods of dosing include “laser” combs using LEDs or laser diodes which must be slowly scanned across the scalp or full-head hoods similar in appearance and dimensions to the classic hair salon hair dryer hood which deliver red light to the head, usually in a doctor's office setting.

Conventional phototherapy regimens generally require the patient to administer the therapy, either by applying the light themselves, region by region with a light comb or by sitting under a hood in a medical or salon setting.

Another complication of conventional phototherapy is that patient compliance with the therapy requirement of a three times weekly, fifteen minutes per session is not good, largely due to the difficulties described above. Another serious drawback for users with thinning but substantial remaining hair is that their at-risk remaining hair blocks therapeutic light from reaching their scalps, eliminating or substantially reducing the beneficial effects of LLLT.

### SUMMARY

The phototherapy light cap discussed below is a flexible, generally hemispherical cap having a light source to supply suitable energy dosage for light therapy. The phototherapy light cap may also include a rechargeable battery source, a light source or an array of light sources, a light diffuser and an interface to a recharging source that may be a docking station. The phototherapy light cap may alternatively be an insert for any commercial head covering or headgear, so that it may be disguised during use.

An alternate phototherapy apparatus includes one or more light sources such as LEDs or lasers, a power source, and a series of optical fiber strands that originate from the light source and terminate near the scalp of the wearer. The light therapy hat may also include an optical switch, or any suitable switch apparatus, to distribute light from the source to the various optical fibers, a programmable integrated circuit to control the switch, and a flexible polymer matrix to hold the terminal ends of the fibers relative to the scalp. The matrix may be adapted to fit under any suitable hat such as a standard baseball cap. The terminal end of the optical fiber strands may include any suitable 90° termination and or any suitable lens or lenses to control the distribution of the illumination.

The phototherapy light cap may combine phototherapy with one or more of a host of therapies such as vibration, massage, occlusion (creating a warm scalp environment by preventing scalp heat and moisture from escaping), ventilation, heating, cooling and a variety of liquid applications. A method for combined therapy includes the steps of parting the user's hair along the sagittal midline to expose the scalp, and applying hair regrowth formulations (preferably a foam or gel of sufficient viscosity) to the scalp along the sagittal midline, and placing the phototherapy cap over the treated scalp area to deliver illumination, heat & occlusion. This serves to liquefy the hair regrowth formulations and keep volatile solvents such as alcohol trapped, and provides illumination as a hair regrowth agent and to improve transdermal absorption of hair regrowth formulations.

Alternate configurations may also include a microprocessor-controlled light dosing circuit suitable for controlling the scalp area to be treated and associated software and provision of red tracer lights for an infrared dosing apparatus. An alternate configuration includes one or more light guides to convey the therapeutic light energy from the cap surface past any hair to the surface of the scalp. A passive phototherapy light cap may provide filters to eliminate unwanted light energy having non-therapeutic or counter-therapeutic frequencies.

There are energy spectra that facilitate hair follicle growth, each absorption peak with about 20 to 40 nm full spectral width (at half maximum) centered about four key wavelengths in the visible and infrared region of the spectrum, specifically at 630 nm, 670 nm, 800 nm and 900 nm. Typical dosing levels are from 1-10 J/cm², delivered over a duration of ten or more minutes, shorter durations being less effective. Thus, individual light sources, each illuminating a one square centimeter area of scalp with a 5-10 mW total output power integrated over that square cm constitute a suitable light source array.

Combining the use of a phototherapy light cap with gelatinized therapeutic agents provides a suitable treatment technique for a scalp utilizing phototherapy, heat and any suitable combination of active ingredients such as minoxidil in an amount within the range of about 2.5 to 5 percent. A gelatinized therapeutic compound may be easily applied to the scalp by laying the gelatinized strip on an area of the scalp to be treated and placing a phototherapy light cap over the scalp area to be treated. Turning on the phototherapy light cap applies light and heat to the scalp and the therapeutic compound which melts the therapeutic compound causing it to flow over the scalp under the influence of gravity and the light and heat from the phototherapy light cap. Inclusion of a secure band on the edges of the phototherapy light cap will contain the flow of the therapeutic compound.

Any suitable surfactant may be included in the therapeutic compound to optimize the flow of therapeutic compound over the scalp area to be treated. Alternatively, a surfactant may be directly applied only to the scalp area to be treated to enhance the flow of the therapeutic compound into and across the scalp area to be treated and retard the flow of the therapeutic compound to areas of the scalp that are not to be treated.

A method of treating and preventing hair loss of a patient includes the steps of applying a surfactant to an area of the patient's scalp to be treated, and then applying a therapeutic gelatin mixture to a portion of the patient's scalp, the therapeutic gelatin mixture forming at least one gelatin strip having at least a 2.5 percent minoxidil, and then providing a phototherapy apparatus over the therapeutic gelatin mixture on the patient's scalp, the phototherapy apparatus including a power supply, a power controller, a plurality of semiconductor light elements powered by the power supply under control of the power controller, the plurality of light emitting diodes providing phototherapy to a patient's scalp, a membrane for securing and supporting the plurality of semiconductor light elements, the membrane shaped to form a generally hemispherical shape with a concave inner surface with the semiconductor light elements on the concave inner surface, a cap for enclosing the membrane, supporting the power supply and the power controller and for retaining the therapeutic gelatin mixture on the patient's scalp, and operating the phototherapy apparatus to illuminate the patient's scalp and the therapeutic gelatin mixture, and further operating the phototherapy apparatus for a therapeutic period of time after liquefaction of the therapeutic gelatin mixture.

A portable phototherapy apparatus includes a power supply, a power controller, a plurality of semiconductor light elements powered by the power supply under control of the power controller, the plurality of semiconductor light elements providing phototherapy to an area to be treated of a patient's scalp, a membrane for securing and supporting the plurality of semiconductor light elements, the membrane shaped to form a generally hemispherical shape with a concave inner surface with the semiconductor light elements on the concave inner surface; and a cap for enclosing the membrane and retaining the therapeutic gelatin mixture on the patient's scalp.

### BRIEF DESCRIPTION OF THE DRAWINGS

FIG. 1A is a top view of a phototherapy light cap.

FIG. 1B is a side view of the phototherapy light cap of FIG. 1A.

FIG. 1C is a cross-section view of the phototherapy light cap of FIG. 1A taken along A-A.

FIG. 2A is a top view of an LED array.

FIG. 2B is a close view of an LED connected to a portion of an array circuit.

FIG. 2C is a schematic diagram of a light module with four series LEDs.

FIG. 3 is a cross-section of an alternate phototherapy light cap.

FIG. 4 is a cross-section of another alternate phototherapy light cap.

FIG. 5 is a top view of a woven conductor array.

FIG. 6 is a top view of a woven conductor array.

FIG. 7 is a cross-section of a phototherapy light cap with light guides.

FIG. 8 is a cross-section of a light guide configuration for high intensity sources.

FIG. 9A is a top view of a beam splitter light distribution system for high intensity light sources.

FIG. 9B is a side view of the beam splitter light distribution system of FIG. 9A.

FIG. 10 is a plan view of a portion of a multilayer phototherapy lattice.

FIGS. 10A, 10B, 10C and 10D are cross-section views of the portion of a multilayer phototherapy lattice of FIG. 10.

FIG. 11 is a plan view of a portion of a combined multilayer phototherapy and liquid treatment lattice.

FIGS. 11A, 11B, 11C and 11D are cross-section views of the portion of a multilayer phototherapy lattice of FIG. 11.

FIG. 12 is a perspective view of a passive phototherapy light cap.

FIG. 13 is a perspective view of a users scalp with a gelatinized therapeutic agent applied and the phototherapy cap ready to cover.

FIG. 14 is a perspective view of the user of FIG. 13 undergoing combined scalp therapy with the cover cap and the illumination insert rendered transparent.

### DETAILED DESCRIPTION OF THE INVENTIONS

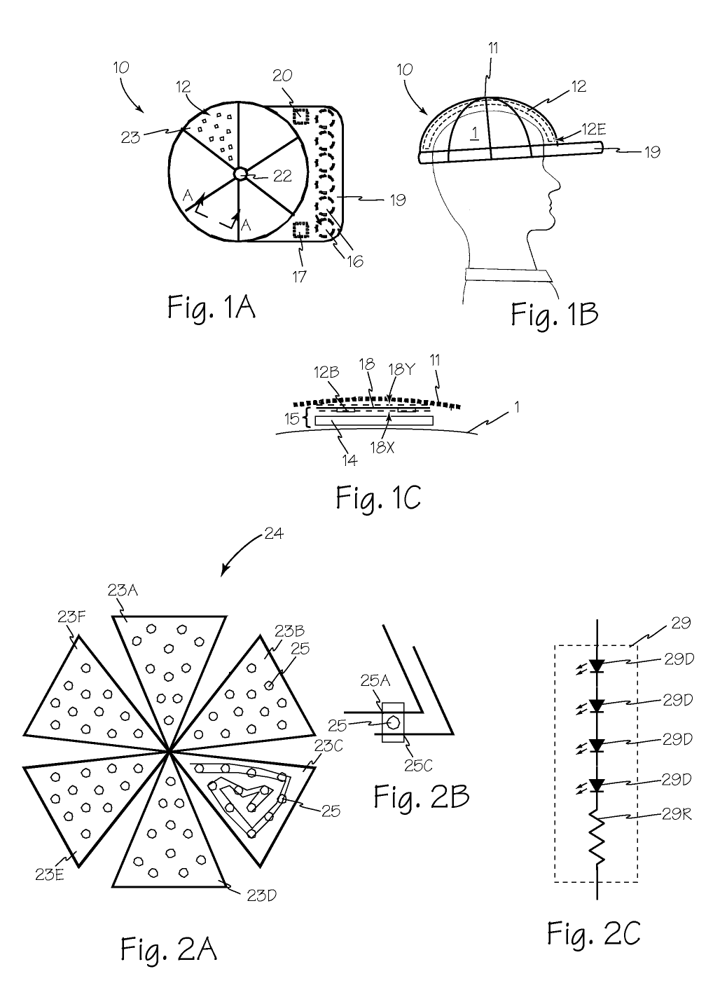

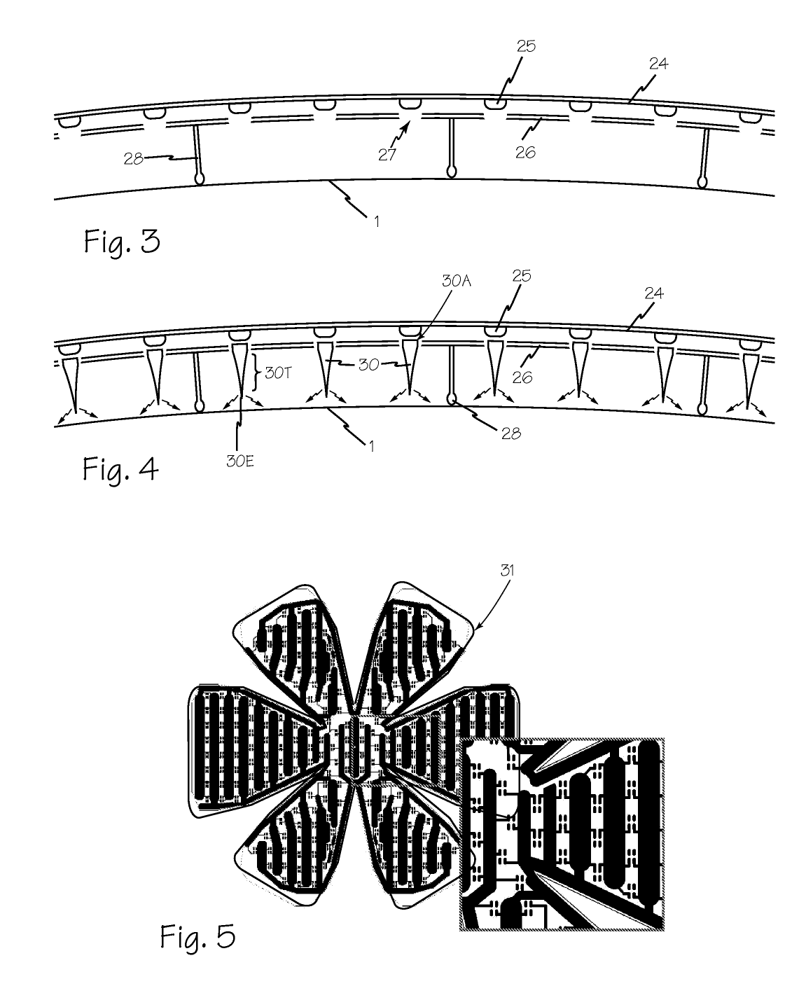

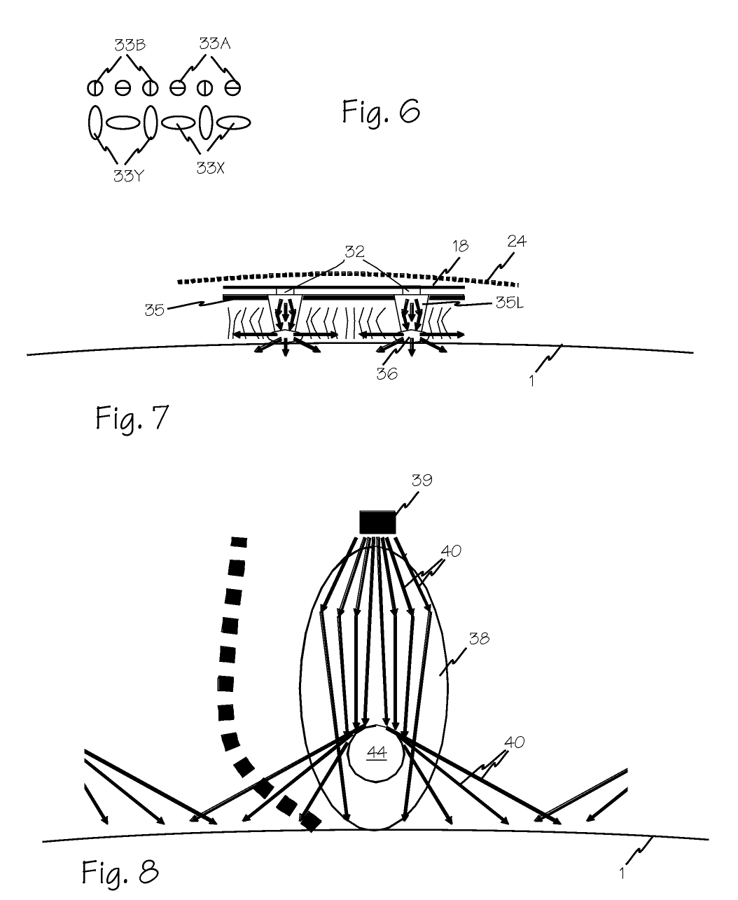

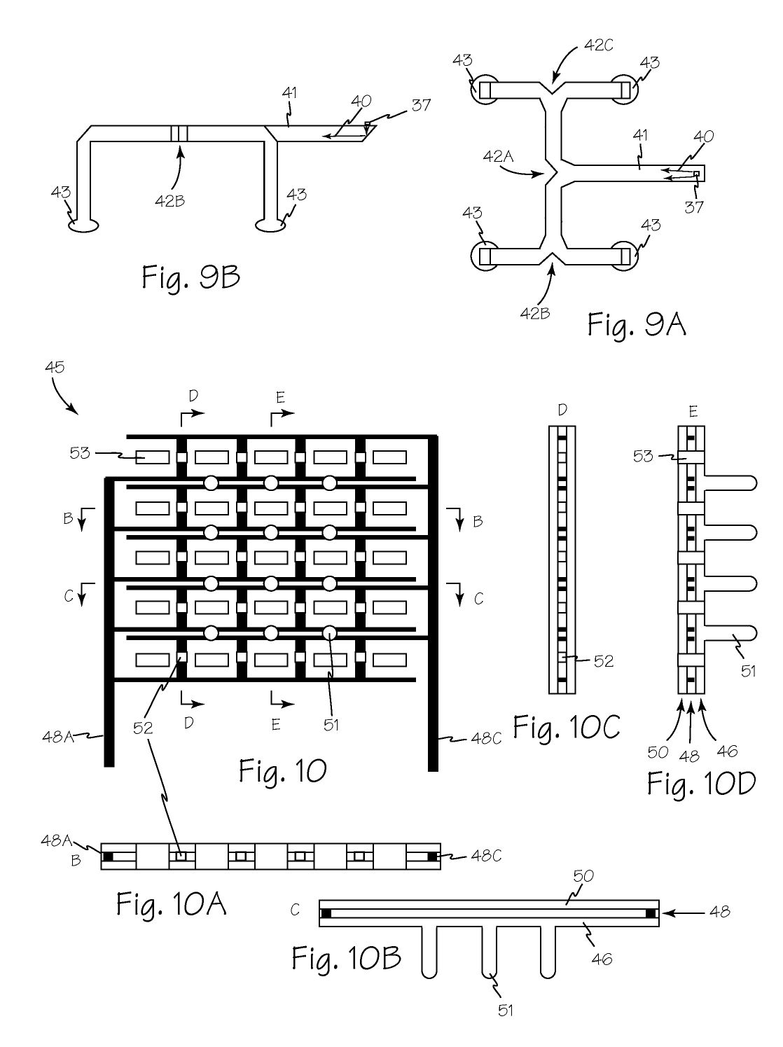

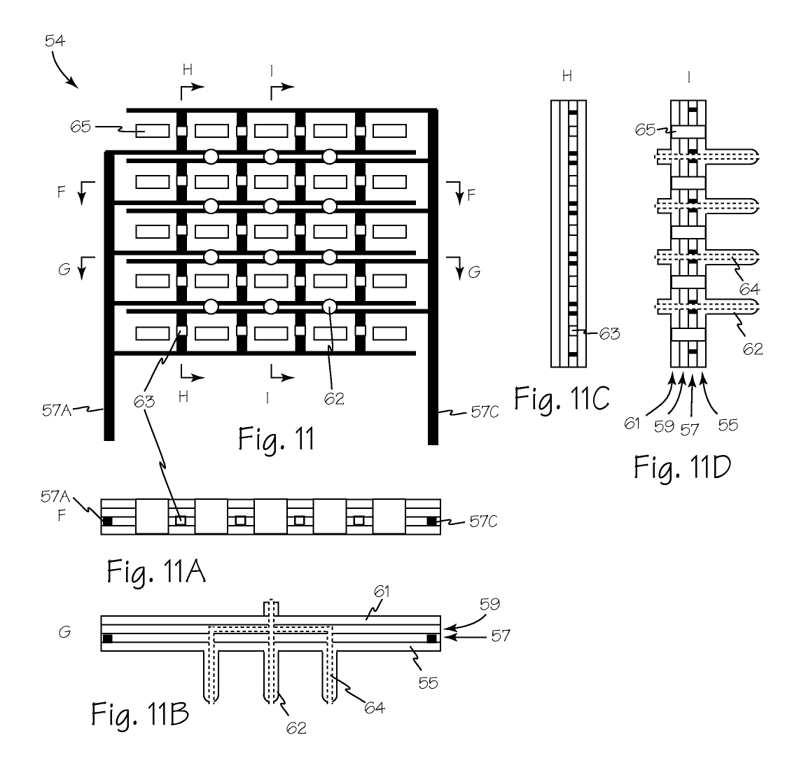

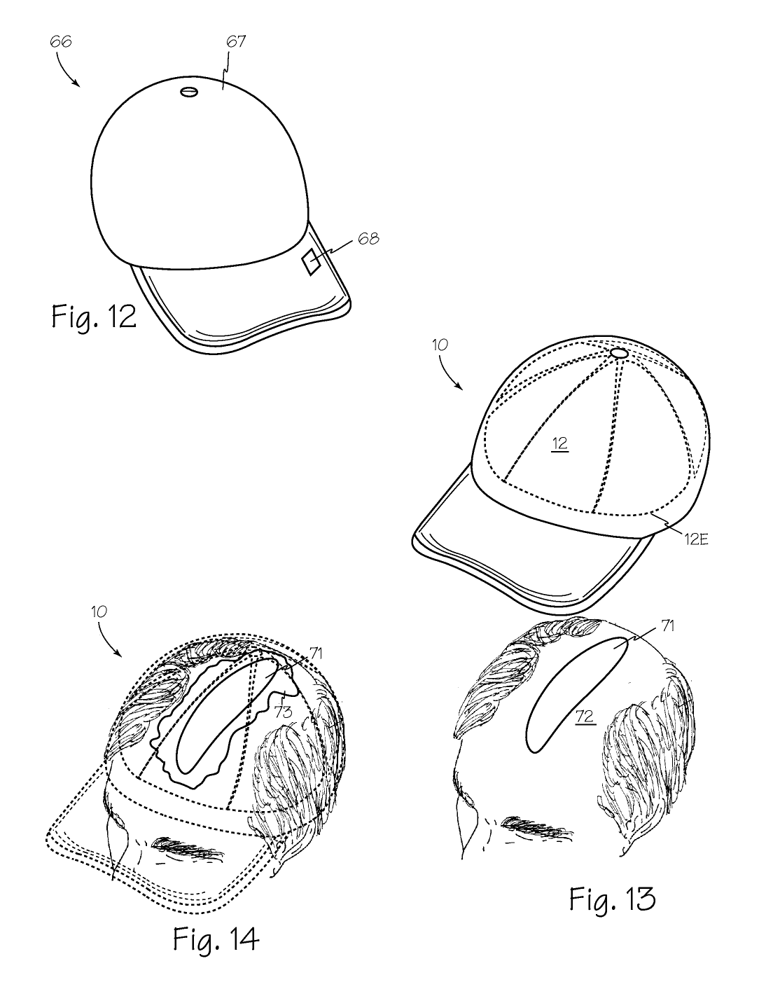

Cap 10 of FIGS. 1A, 1B and 1C includes cover 11 protecting and shielding illumination array 12 and diffuser 14 which combine as therapy insert 15. Cap 10 may be powered by one or more rechargeable batteries such as batteries 16 and controlled using switch 17 which may be secured to hat brim 19 along with microcontroller or microprocessor 20. Batteries 16 may be recharged through a button connector/interface such as connector 22. The power source, power controller and switch may also be separated from the therapy insert and provide the electrical power through any suitable tether.

As shown in FIG. 2A, the phototherapy insert includes several triangles, segments or gores 23A, 23B, 23C, 23D, 23E and 23F that may be secured at their edges to form a generally hemispherical cap. The illumination arrays in each gore such as array 12 are generally identical although they may have some differences such as one segment powering or controlling one or more slave segments. Each illumination array may be controlled simultaneously or separately and may each consist of subarrays under separate control. Individual light elements such as light element 25 may be any suitable solid state or semiconductor light element or generator such as an LED, OLED, semiconductor or laser diodes, solid state laser and the like. Independent area control is desirable for clinical trial studies or to deliver phototherapy differentially to different areas of the scalp.

FIG. 2A and FIG. 2B illustrate a configuration of illumination elements for each gore, such as gores 23A, 23B, 23C, 23D, 23E and or 23F. The illumination arrays may be wired in series as illustrated with all the anodes 25A connected in common and all cathodes 25C connected in common. Alternatively, light modules such as light module 29 of FIG. 2C may be connected between anode 25A and cathode 25C. Within each light module are four elements 29D in series with a current limiting resistor 29R. This configuration permits the voltage to each light module to be higher but the total current to be lower, resulting in reduced resistive heating and thus less wasted power. Series resistor 29R prevents catastrophic failure of the unit by limiting current to a value tolerable by a single light element such as laser diode 29D.

Each gore, petal, wing or sector such as gore 23A may be separate elements and may be separately controlled. Thus if gores 23A, 23B and 23C are located on the right side of a cap and gores 23D, 23E and 23F are located on the left side of a cap, and gores 23A and 23F are in the front of a cap, then gores 23C and 23D are in the back of the cap. With this configuration, typical male pattern baldness may be treated using primarily gores 23C and 23D, and typical female front-centered baldness may be treated using primarily gores 23A and 23F. With specific sector control each sector may be independently controlled for time and or intensity. Different gores may also include different light elements such as primary gores 23A and 23F having laser diodes and secondary gores include LEDs. Similarly the light elements may be structurally similar such as all LEDs and the light elements in different gores may be selected to produce light of different frequencies.

The configuration of flexible substrates such as flexible substrate 18 used to form gores, petals, wings or sectors selected to form a phototherapy light cap may be arranged in the desired configuration and encapsulated together using inner encapsulation layer 18X and outer encapsulation layer 18Y with both inner and outer encapsulation layers sealed along periphery 12E.

The phototherapy light cap is discussed with respect to red-light phototherapy dosing but it can be used to apply any other wavelength optical therapies. For example, the cap light dispenser can be used to deliver broad-spectrum white light therapy, which is preferred by some users. Commercially available white light LEDs consisting of blue LEDs with phosphor layers will provide the desired intensity, up to and beyond typical bright daylight brightness levels. The current therapeutic cap or cap insert concept can also be extended to heat or cooling inserts or electromagnetic therapies. The phototherapy light cap may also be constructed so as to provide any combination of these and other therapies at the same time.

Power and or temperature control may be managed through control of the duty cycle of the light elements, light modules, gores, petals, wings or sectors. Controller 20 may be used to control the duty cycle of, for example lasers, to minimize internal heating and thus optimize light output.

The phototherapy light cap may additionally combine one or more of a host of therapies such as vibration, massage, occlusion (creating a warm scalp environment by preventing scalp heat and moisture from escaping), ventilation, heating, cooling and a variety of liquid applications. For example, a combined therapy approach for hair regrowth would include the steps:
1. part user's hair, as necessary, along the sagittal midline exposing the scalp;
2. apply a hair regrowth formulation to the scalp along the sagittal midline, using a foam, gel, or gelatinized strip;
3. place phototherapy cap over the treated scalp area to deliver heat & occlusion to liquefy the hair regrowth formulations and keep volatile solvents (e.g., alcohol) trapped while providing heat and illumination as a hair regrowth agent and to improve transdermal absorption of formulations.

Alternatively, a combined therapy approach for hair regrowth where hair is absent would include the steps:
1. apply a hair regrowth formulation to the entire affected head (front, top, crown, occipital);
2. work slightly into hair;
3. place phototherapy cap over the treated scalp area to deliver heat & occlusion to liquefy the hair regrowth formulation and keep volatile solvents (e.g., alcohol) trapped while providing illumination as a hair regrowth agent and to improve transdermal absorption of formulations.

In both examples, the hair regrowth formulations may also include elements for hair volumizing and or camouflage with keratin-like powder or scalp dye which may be heat-activated and/or heat-cured substances, light-activated and/or light-cured substances. The phototherapy cap may then deliver heat & illumination to physically and or chemically change cosmetic formulations to provide a suitable cosmetic effect.

Referring again to FIG. 1C, a first configuration suitable for very thin hair in which the hair does not present a significant light shield to the scalp, a high-forward scatter diffuser such as diffuser 14 may be used in close proximity to illuminators such as light emitting diodes 12B, achieving uniform illumination and requiring only very small separation between scalp and flexible substrate 12A. Diffusers can range from inherently scattering, usually milky-colored plastics to dielectric-scatterer impregnated plastics and standard photographer's white diffuser cloth.

The diffuser in a thin-film diffuser configuration may be a photographic plastic forward scattering film, for example, folded over as necessary to create a suitable diffuser. The complete power rail, LED, diffuser combination may have a thickness of under 5 mm. Various high forward scattering materials and engineered materials can be used to create an optimally thin and effective diffuser. The diffuser must have minimum attenuation of light while angularly spreading the preferably wide-angle LED output beam further out so that the diffuser output illuminates the scalp with uniform dosage. The goal is to have the diffuser appear as a uniformly glowing forward light emitter.

Referring now to FIG. 3, when the hair is thin enough to be insubstantially opaque to LLLT illumination, uniform scalp illumination may be achieved by spatial separation of an array of suitable illuminators such as wide-angle emission surface-emitting LEDs combined with use of a light emitter-scalp separation layer, preferably consisting of bristles under laying the LED array in a suitable pattern to maintain adequate separation for the individual LED beams to diverge adequately to achieve uniform illumination. Illumination substrate 24 supports one or more illuminators such as LED 25. Spacer 26 includes a plurality of openings 27 oriented relative to the illuminators to optimize the light energy emissions of the illuminators and to uniformly illuminate scalp 1. Spacer 26 includes one or more bristles such as bristle 28 to maintain a predetermined space between illuminators 25 and scalp 1.

Wide-angle LEDs can be used without a diffuser by employing a short separation between the LED array and the scalp surface to allow the individual beams to diverge enough to achieve uniform intensity surface illumination. Experiments were performed using a photodiode linearized in a transimpedance configuration to measure absorbed energy with respect to position at various heights above the LED array. With only 6 mm of separation, the beam varied 20% across the illuminated flat surface. With 9 mm spacing, the variation was under 3%. A simple LED array with 5 mm to 10 mm spacer bristles will provide adequate uniformity for the scalp.

Another alternate technique for achieving uniform illumination is by redirecting the therapeutic light energy from the source to points under most of an opaque hair volume by means of waveguides. Referring now to FIG. 4, one or more waveguides such as waveguide 30 may employ a design that has a wide-diameter LED-side surface 30A to ease alignment tolerance combined after an adiabatic taper 30T, to a smaller waveguide portion that emits a wide-angle emission from emitting surface 30E. Additional spacer bristles may also provided for the “hair bypass” light guides to achieve uniformity.

As illustrated in FIG. 5, the light source arrays can be placed at grid points in a woven conductor array such as conductor array 31 of FIG. 5 or on a flexible PC board. This single-layer board consists of a copper interconnect layer sandwiched between two thin polyimide layers.

When illuminators are lasers and used with little or no diffusion, orientation of the illuminators must be controlled. Referring now to FIG. 6, Laser illuminators 33A and 33B produce elliptical output fields 33X and 33Y respectively depending on the orientation of the laser illuminator. Any suitable pattern of laser illuminator orientations may be used to achieve therapeutic results. For example, alternating orientations with a 90 degree difference as illustrated in FIG. 6.66

Referring now to FIG. 7, LED array 32 illuminates a co-registered lens and waveguide array 35 that directs light inwardly toward the scalp through a light guide 35L that penetrates the hair and terminates at the scalp 1, in contact with the scalp. At the scalp level, light energy from the light guide is dispersed so as to re-emit in all downward directions, bathing the scalp with substantially uniform illumination, bypassing the hair above it.

Scattering bulb 36 in contact with the scalp can be, for example, a single or multiple reflective and or refractive elements, preferably spherical, that will refract and or reflect light with little loss but redirect it around the bulb so that it more or less uniformly illuminates the scalp.

Referring now to FIG. 8, another light guiding configuration such as light guide 38 employs refraction and reflection to conduct therapeutic light past hairs such as hair 34 to the scalp. Light source 39 which may be LEDs or other suitable light sources such as lasers. Light 40 is captured by an integrated, preferably plastic, waveguide channel 38 which collects and focuses light 40 from the source and then redirects it downward toward an included scatterer 44. The downward light is then uniformly directed toward the scalp.

Referring now to FIGS. 8A and 8B, high intensity light source 37 may be LEDs or other suitable light sources such as lasers. Light 40 is captured by an integrated, preferably plastic, waveguide channel 41 which collects and repetitively splits the light 40 using splitters 42A, 42B and 42C and then redirects it downward to the hair at a plurality of re-emitter locations such as diffuser 43. The downward light can be directed to the scalp, to a diffuser or to a subsequent additional waveguide element.

Waveguides for the hair-bypass configurations of the LED array may be constructed from flexible 1 mm diameter acrylic rods. The emission at 9 mm spacing from the rod array ends will be uniform. Alignment tolerance is tight for 1 mm diameter rods and the output divergence angle is somewhat smaller than the original LED sources. Both of these limitations can be advantageously traversed by use of tapered rods that have a larger emitter-side diameter, such as 2 mm, and a smaller output diameter, typically 0.5 mm.

Although there are many ways of generating light in the desired wavelength bands at the desired total power levels, such as by fluorescent, incandescent, laser diode, LED and photo luminescent sources, a suitable method of delivering light is by means of an array of light emitting diodes, preferable because they meet the optical power requirements while being low in operating voltage and electrically efficient, low in cost, have a wide emission angle and are therefore able to illuminate a wide area more or less uniformly with a relatively thin intervening diffuser. Fewer diodes or a single diode source may be able to have their output directed to emanate quite uniformly from a broad surface, but the sources represent a concentrated heat load and light source and are burdened by the light distribution requirement.

Surface emitting light emitting diodes are currently preferred because they are low in profile and can emit light with very little source footprint either in area or in thickness. Thin diffuse light-source inserts are preferred so that the overall therapeutic device is as comfortable to wear and as unobtrusive as possible. The ideal device is battery powered (as by rechargeable battery embedded or tethered to the therapy insert). Suitable rechargeable matrices include lithium ion polymers which possess an energy density of over 100 Watt-hours per kg. For example, to achieve full adult head coverage using 10 J/cm2 over 300 cm2 would require 3 kJ of energy. With a 20% electrical-to-optical conversion efficiency, 15 kJ of stored energy per dosing would be required, which is equivalent to less than 5 Wh of battery storage capability or 50 grams weight of storage medium. A typical baseball cap weighs about 80 grams, so that the insert itself, including battery, connection matrix, light source(s) and light diffuser need not more than double the typical cap weight.

Although the illumination array is not likely to produce uncomfortable heat levels on the scalp, it is possible to move heat load elsewhere on the cap or to the outer surface of the cap by various electrical or passive heat conducting means. Cooling can be achieved by Peltier cells, if desired, heat being dissipated in the brim or outer surface of the cap. Creating an array of ventilation holes for convective cooling may also be achieved with no significant reduction of the light intensity directed toward the scalp.

FIG. 9 illustrates a portion of a multilayer phototherapy lattice 45 formed of substrate layer 46, opto-electronics layer 48 and capping layer 50. Substrate layer 46, which is the scalp side layer may also includes an array of bristles such as bristle 51. Opto-electronics layer 48 includes flexible conductor arrays such as anode array 48A and cathode array 48C as well as LEDs such as LED 52 which is soldered or otherwise electrically connected between anode array 48A and cathode array 48C. Capping layer 50 creates a hermetic seal for opto-electronics layer 48. The multiple layers and or bristles may be formed of any suitable flexible material such as silicone and may formed in any suitable color or be clear. Lattice 45 also includes ventilation openings such as vent opening 53 to reduce weight and provide good ventilation through the lattice.

In an alternative multilayer therapy lattice of FIG. 10 multimode multilayer therapy lattice 54 includes substrate layer 55, opto-electronics layer 57, fluid distribution layer 59 and capping layer 61. Substrate layer 55, which is the scalp side layer may also includes an array of bristles such as bristle 62. Opto-electronics layer 57 includes flexible conductor arrays such as anode array 57A and cathode array 57C as well as LEDs such as LED 63 which is soldered or otherwise electrically connected between anode array 57A and cathode array 57C. Fluid distribution layer 59 includes multiple interconnected fluid distribution lumens or channels 64 for simultaneous delivery of therapeutic fluids, foams, compounds and or formulations to assist in low-level light therapy or as simultaneous therapy. Capping layer 61 creates a hermetic seal for fluid distribution layer 59 and opto-electronics layer 57. The multiple layers and or bristles may be formed of any suitable flexible material such as silicone and may formed in any suitable color or be clear. Lattice 54 also includes ventilation openings such a vent opening 65 to reduce weight and provide good ventilation through the lattice.

Referring now to FIG. 11, passive light therapy cap 66 may be used to filter out external light sources (room light or sunlight) so as to provide only therapeutic wavelengths to the patient's scalp. Passive phototherapy will generally involve intense “white-light” sources, such as sunlight, which is not effective in hair growth therapy because the green and blue regions of the visible spectrum are deleterious at high intensities. Therefore, sunlight therapy may be achieved by passing only therapeutic wavelengths to the scalp. Such a configuration of restricted-wavelength phototherapy may be used because it is passive and the light source of choice, daylight, is free and ubiquitous. Such a passive cap can be configured to also prevent ultraviolet exposure.

Achieving wavelength-specific attenuation is possible in a variety of ways. For example, filter element 67 colored films consisting of a polymer matrix with various dyes can achieve red-only transparency. More sophisticated multilayer dielectric films can provide reflectivity in blue and green portions of the spectrum to provide relative cooling. Such films, which can also be comprised to absorb rather than reflect unwanted wavelengths, can be tailored to have complex spectral shapes for more demanding wavelength-specific therapy or novelty purposes.

The red wavelength region of the solar spectrum is intense enough to provide adequate dosing (10 J/cm²) over a ten to thirty minute period. Because different cloud cover, times of year, geographical locations, etc will alter the optical power level, this technique optionally provides for a resettable dosimeter element 68 which lets the user know when they have achieved a selected degree of exposure.

Referring now to FIG. 13, phototherapy cap 10 is illustrated with illumination array 12 as discussed above. To achieve combined scalp therapy, a user 70 applies gelatinized therapeutic strip 71 to one or more portions of scalp to be treated such as crown 72. After placement of one or more gelatinized therapeutic strips such as strip 71, user 70 secures cap 10 covering the area to be treated and energizes all or a portion of illumination array 12 as shown in FIG. 14.

The frequency of the illumination and the heat generated by illumination array 12 and the scalp melt the therapeutic strip[s] and the therapeutic agent and liquid gelatin mixture 73 flows over the area to be treated.

Therapeutic strips or gelstrips such as strip 71 may be prepared by combining one part of gelatin with ten parts water to prepare a gelatin base for any suitable therapeutic compound such as minoxidil. The one part to ten parts composition may be produced with one part gelatin to three parts cold water soaking together for about one minute. Then, the other seven more parts water can be added and heated to about 120 degrees Fahrenheit. The resulting gelatin base composition may cool until it generally begins to solidify or gel.

The cooling gelatin base is then added to an approximately equal volume amount of five percent minoxidil solution to make a therapeutic mixture which is approximately 2.5 percent active ingredient. Pour the resulting therapeutic mixture into any suitable mold such as two milliliter rectangular molds or generally larger rectangular strips. The rectangles or strips may be further formed into any desirable size and shape for distribution, application, or further processing. Any suitable shape may be adopted for the therapeutic gelatin mixture to optimize application of the therapeutic component to the scalp or other area of a users body.

Therapeutic gelatin mixtures may also be employed to deliver other chemical entities which stimulate the scalp and/or prepare the scalp for hair growth medicaments. Therapeutic gelatin mixtures may also incorporate pectin, polysaccharides, fatty acids, gelling agents, excipients, solutions, emulsions, encapsulants, microspheres, or the like. Additional surfactants may be added to optimize the flow characteristics of the gelatin mixture as it is heated. Active ingredients may include: topical finasteride, minoxidil, ketoconazole, steroids, other anti-microbials, steroids, copper peptides for post-hair transplantation wound healing, anti-androgens, antimicrobials, spironolactone, spironolactone-like compounds, progesterone derivatives, betametazone valerate, ketoconazole, zinc salts, Zinc Pyrithione ZnP (head and shoulders), finasteride, flutamide, dutasteride, melatonin, photo-activated compounds, lice treatments, cosmetic preparations such as scalp dye, hair dye, hair gel, conditioner, moisturizer, scalp oils, hair “volumizers,” vitamins, minerals, herbals, therapeutic water, zinc, iron, biotin, folic acid, anti-androgens, tretinoin, azelaic acid, and saw palmetto. The preparations may be provided in liquids of various viscosity, or in foams or other fluids, slurries or suspensions.

The firmness of the gelatin strips and the rate at which the gelatin melts can be regulated by increasing or decreasing the amount of gelatin or other ingredients such as ethanol or other surface tension solvents. Where the five percent minoxidil solution includes ethanol, propylene glycol and the like, the relative percentages of those components may also be varied as desired. The amount or percentage of water may also be varied.

Coloring may also be included in the therapeutic gelatin strips. For example, patients with darker hair, a colorant may be added to allow the therapeutic gelatin mixture to blend in with the user's hair color or to operate as camouflage for the user's scalp. In addition to the therapeutic gelatin mixture being applied directly to a scalp, it could also be applied from a bottle, via applicator, or any other suitable method.

Placing a therapeutic gelatin strip on the sagittal midline of the scalp offers an advantage of allowing the gelatin to melt and conduct the active ingredient over successive outer portions of the scalp. A headband or other barrier may also be employed around the perimeter of the skull or the area to be treated such as area 72. The headband would be operative to prevent or reduce seepage of liquefied gelatin and active ingredient down the back of the neck or into the face of the user.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A portable phototherapy apparatus comprising:
a power supply;
a power controller;
a plurality of semiconductor light elements powered by the power supply under control of the power controller, the plurality of semiconductor light elements providing phototherapy to an area to be treated of a patient's scalp;
a membrane for securing and supporting the plurality of semiconductor light elements, the membrane shaped to form a generally hemispherical shape with a concave inner surface with the semiconductor light elements on the concave inner surface; and
a cap for enclosing the membrane and retaining the therapeutic gelatin mixture on the patient's scalp.

2. The portable phototherapy apparatus of claim 1 further comprising:
a plurality of bristles disposed on the concave inner surface of the membrane for creating a predetermined separation between the membrane and the patient's scalp.

3. The portable phototherapy apparatus of claim 2 further comprising:
a plurality of fluid distribution lumens disposed within the plurality of bristles and the membrane for delivering therapeutic compounds to the patient's scalp during phototherapy.

4. The portable phototherapy apparatus of claim 1 further comprising:
a diffuser oriented between the membrane and the patient's scalp.

5. The portable phototherapy apparatus of claim 1 wherein the plurality of semiconductor light elements are LEDs.

6. The portable phototherapy apparatus of claim 1 wherein the plurality of semiconductor light elements are laser diodes.

7. The portable phototherapy apparatus of claim 1 further comprising:
a portion of therapeutic gelatin mixture applied to the area to be treated.

8. The portable phototherapy apparatus of claim 7 wherein the portion of therapeutic gelatin mixture comprises:
one or more therapeutic gelatin strips.

9. A method of treating and preventing hair loss of a patient comprising the steps:
applying a therapeutic gelatin mixture to a portion of the patient's scalp;
providing a phototherapy apparatus over the therapeutic gelatin mixture on the patient's scalp, the phototherapy apparatus including:
a power supply;
a power controller;
a plurality of semiconductor light elements powered by the power supply under control of the power controller, the plurality of light emitting diodes providing phototherapy to a patient's scalp;
a membrane for securing and supporting the plurality of semiconductor light elements, the membrane shaped to form a generally hemispherical shape with a concave inner surface with the semiconductor light elements on the concave inner surface; and
a cap for enclosing the membrane, supporting the power supply and the power controller and for retaining the therapeutic gelatin mixture on the patient's scalp;
operating the phototherapy apparatus to illuminate the patient's scalp and the therapeutic gelatin mixture, and further operating the phototherapy apparatus for a therapeutic period of time after liquefaction of the therapeutic gelatin mixture.

10. The method of treating and preventing hair loss of claim 9 wherein the step of applying a therapeutic gelatin mixture to a portion of the patient's scalp further comprises the steps:
parting a patient's hair exposing the scalp;
applying a therapeutic gelatin mixture to the scalp along the part.

11. The method of treating and preventing hair loss of claim 9 wherein the a therapeutic gelatin mixture further comprises:
heat-activated substances to improve hair volume.

12. The method of treating and preventing hair loss of claim 9 wherein the a therapeutic gelatin mixture further comprises:
heat-activated substances to camouflage hair loss.

13. The method of treating and preventing hair loss of claim 9 wherein the a therapeutic gelatin mixture further comprises:
light-activated substances to improve hair volume.

14. The method of treating and preventing hair loss of claim 9 wherein the a therapeutic gelatin mixture further comprises:
light-activated substances to camouflage hair loss.

15. The method of treating and preventing hair loss of claim 9 wherein the a therapeutic gelatin mixture is at least a 2.5 percent minoxidil mixture.

16. The method of treating and preventing hair loss of claim 9 wherein the step before applying a therapeutic gelatin mixture to a portion of the patient's scalp is the step:
applying a surfactant to an area of the patient's scalp to be treated.
